(19) United States
(12) Patent Application Publication
(10) Pub. No.: US 2020/0395114 A1
(43) Pub. Date: Dec. 17, 2020

(54) Algorithmic Method to Detect Discrepancies in Electronic Medication Histories

(71) Applicant: Avinash Bachwani, Schenectady, NY (US)

(72) Inventor: Avinash Bachwani, Schenectady, NY (US)

(21) Appl. No.: 16/439,724

(22) Filed: Jun. 13, 2019

(51) Int. Cl. G16H 20/10; G16H 10/60; G06F 17/27

(52) U.S. Cl. CPC G16H 20/10; G06F 17/2705; G06F 17/2765; G16H 10/60

(57) ABSTRACT

Medication errors are a leading cause of morbidity and mortality, and impose a tremendous economic and medico-legal burden on society. Discrepancies in medication information across healthcare settings and dispensing pharmacies result in medication errors that are potentially preventable. Method(s) and process(es) using machine-based Boolean logic are described herewith to detect discrepancies in electronic medication histories as effectively as highly trained clinical pharmacists, but with far greater efficiency and parsimony. The reduced cognitive burden and time spent in reconciling discrepant medication information is expected to yield cost savings. Furthermore, if deployed uniformly, the process of reconciling medications electronically may be standardized across Electronic Health Record (EHR) platforms and healthcare settings, resulting in an improvement of medication safety and reduction in medication errors.

CROSS-REFERENCE TO RELATED APPLICATIONS

None.

### FIELD OF THE INVENTION

An algorithmic embodiment relating to healthcare in general, and without limitation, more particularly to the improvement of medication safety and reduction of preventable medication errors due to discrepant medication information across healthcare settings.

### BACKGROUND OF THE INVENTION

Medication errors are pervasive across healthcare settings and constitute the single largest group of medical errors (Classen & Metzger, 2003). While the root cause of any medical error is likely to be multifactorial and complex, most medication errors result from discrepancies in medication information documented across healthcare settings and dispensing pharmacies, and are therefore preventable (Cohen, 2007). Despite being under-reported, medication errors occur with distressing frequency. Medication errors lead to more than 700,000 emergency visits and 100,000 hospitalizations annually. Upon hospitalization, 85% of patients were noted to have at least one error in their medication histories (Gleason et al., 2010). Medication errors are estimated to directly cost the healthcare system 3.5 billion dollars annually, with additional indirect costs being incurred from lost productivity and liability claims (Agency for Health Research and Quality, 2015; Kohn, Corrigan, & Donaldson, 2000).

Medication reconciliation is the process of compiling an accurate and complete list of the most current prescription and over-the counter medications a patient is actually taking, and reconciling discrepant information documented across healthcare settings including medication names (chemical, brand, and generic) and signature information (dosage, frequency and route of administration). This reconciled list of medications informs clinical decisions and prescription actions (renewals, additions, discontinuation or modifications), and also prevents medication errors and adverse events due to unintentional duplications, additions, omissions and discontinuations (Barnsteiner, 2008; Greenwald et al., 2010; Manno & Hayes, 2006; Varkey, Cunningham, & Bisping, 2007). Since 2005, medication reconciliation has been declared a National Patient Safety Goal (NPSG), and is mandated by several federal and state initiatives to be performed at every transition of care (Joint Commission, 2006; Institute for Healthcare Improvement, 2016).

Historically, clinicians performing medication reconciliation would need to assimilate information manually from disparate records across healthcare settings and dispensing pharmacies. In recent years however, Electronic Health Record (EHR) technology and interoperability standards have made it possible for clinicians to electronically import a list of medications prescribed from participating pharmacies and Pharmacy Benefit Managers (PBMs) as electronic medication histories (Gabriel and Swain, 2014; Surescripts, 2019). The ability to import electronic medication histories has been accompanied by several innovative approaches to reconcile medications electronically that can be classified broadly into two categories—(a) non-EHR approaches: tools outside the EHR that aggregate medication information from disparate sources and present medication list(s) for reconciliation including web-based tools, and (b) EHR-based approaches: ranging from check-in kiosks for patients to specific EHR functionality (e.g. side-by-side display of home and current medications, duplicate alerts, ability to sort alphabetically, chronologically, by prescriber, by encounter etc.). Some tools incorporate Natural Language Processing (NLP), Machine Learning (ML), and Collaborative Filtering (CF) techniques, or a combination thereof to avoid omissions and add contextual and temporal relationships to prescriptions from medication lists and clinical corpora (Cimino, Bright, & Li, 2007; Uzuner, Solti, & Cadag, 2010; Jagannathan et al., 2009, Li, Liu, Antieau, Cao, & Yu, 2010, Hasan, Duncan, Neill, & Padman, 2011). Generally, non-EHR approaches do not integrate within clinical workflows since medications are reconciled outside the EHR, while EHR-based approaches often impose a cognitive burden on clinicians due to the volume of medication entries (e.g. multiple entries, formulary substitutions) and discrepant signature information from various prescribers and dispensing locations (e.g. dosing variations among prescribers, free-text fields, incomplete signature information). Both approaches lack generalizability beyond their native environments. Please see non-exhaustive tabular summary of patented and non-patented, EHR and non-EHR approaches to reconcile medications electronically in Appendix A.

The average time taken to reconcile medications is 15 minutes (range 1-75 minutes) and costs approximately $30 per patient depending on healthcare setting, interviewing skills, and patient participation (Gleason et al., 2004; Schenkel, 2008). Some healthcare organizations dedicate clinical pharmacists for medication reconciliation, often considered as the gold-standard, while others utilize nurses, pharmacy technicians, interns, residents, and physicians, or a combination thereof, further influencing the time, cost, and quality of reconciliation (Schnipper et al., 2009; Lesselroth et al., 2009).

### BRIEF SUMMARY OF THE INVENTION

A method and process to improve medication safety and reduce medication errors, by standardizing the operation to detect discrepancies in electronic medication histories and presenting the processed medication list in standard categories of discrepant and non-discrepant medication in the electronic medication history response across EHR platforms and healthcare settings, effectively reducing the cognitive burden and time involved in reconciling discrepant medications.

### BRIEF DESCRIPTION OF THE DRAWINGS AND APPENDICES

FIG. 1 is a schematic diagram of one illustrative embodiment of the algorithm that depicts how the algorithm may he incorporated into clinical workflows to identify discrepancies, and the estimated net impact incorporating the algorithm may have on the process of medication reconciliation;

FIG. 2 is a flowchart of one illustrative embodiment of the algorithm that depicts how the electronic medication history may be processed, the machine-based (Boolean) logic used to identify and categorize discrepancies, and the format used to present the medication list to promote medication safety and reduce medication errors;

Appendix A: Tabular summary of various patented and non-patented, EHR and non-EHR approaches to reconcile medications electronically;

Appendix B: Program code of one illustrative embodiment of the algorithm that processes the electronic medication history, identifies and categorizes discrepancies, and presents the medication list in accordance to one embodiment that promotes medication reconciliation and reduces medication errors;

Appendix C: References; and

Appendix D: Glossary (of terms).

### DETAILED DESCRIPTION OF THE INVENTION

The formerly resource intensive task of assimilating medication information from disparate records across healthcare settings and dispensing pharmacies has already been largely curtailed by the ability to import electronic medication histories into an EHR as outlined above [0004]. In processing the imported electronic medication history through the algorithm depicted in FIG. 1, each entry contained therein is initially verified to be an actual medication by its standard numeric drug identifier. Then immunizations and supplies are categorized as such, and entries that either lack standard numeric drug identifiers or those that have been free-texted are categorized as non-standard discrepancies. Subsequently, duplicate entries including partial string matches and/or evolving signature information (e.g. dosing change—Tylenol® 325 mg twice a day changed to Tylenol® 650 mg twice a day) are categorized as duplicate discrepancies. Hereafter, all non-discrepant, unique medications are categorized as either acute or chronic, depending on whether the prescription was issued for greater than 30 days and/or refills authorized. Acute medications are categorized separately since short-term medications seldom need to be continued at subsequent clinical encounters. Based on this algorithm, a Boolean program was created using CSC (Microsoft® Corporation) with Microsoft® Visual Studio Professional 2013 (version 12.0.31101.00, update 4) on the Microsoft®.NET framework (version 4.5.51209) as described in Appendix B. The processed and categorized medication list (output) comprising of discrepant (non-standard entries, immunizations and supplies, and duplicate) and non-discrepant acute and chronic medications can then be presented for reconciliation as part of the electronic medication history response within the native EHR without any impact on clinical workflows as depicted in FIG. 2.

After obtaining approval from the Institutional Review Board of a community hospital, a retrospective sample of 384 records containing electronic medication histories reconciled by a clinical pharmacist during the admission of adult patients from the Emergency Department over an eight-week period between October and November 2016 were processed through this algorithm. The discrepancies identified by the algorithm were compared with those identified by the clinical pharmacist, and analyzed using SPSS® Statistics (version 24; International Business Machines). Since the algorithm uses machine-based Boolean logic to identify discrepancies, while clinical pharmacists utilize clinical experience and heuristics based on a priori knowledge and training (Vogelsmeier, Pepper, Oderda, & Weir, 2013), an assumption of independence was made. An independent samples t test showed that there was no significant difference in the number of discrepancies identified by the algorithm (m=10.89, s=9.649) and clinical pharmacist (m=11.26, SD=9.657), t(766)=0.531, p=0.596. Furthermore, the algorithm identified the same number of duplicate (10) and non-standard (0.63) discrepancies as the clinical pharmacist.

All 384 records were processed in 68 seconds using an Intel© Core™ 2^{nd} generation i5 dual-core processor (i5-2430M) with a Processor Base Frequency of 2.4 GHz and 4 GB installed Random Access Memory (RAM). The mean processing time was 26.84 milliseconds per record (minimum 0.0475 milliseconds, maximum 281.30 milliseconds). The low-level consumption of computing resources for a linear polynomial-time function such as this portends well for general deployment since the output may neither be significantly influenced by the length of the input string (number of medications), nor the variable processing speeds at the point of care delivery.

Analysis of medication orders placed by physicians at admission based on the reconciled medication lists generated by the clinical pharmacists identified six medication errors (1.56%). Case studies and expert opinion revealed that none of the medication errors resulted from discrepancies persisting after medication reconciliation, but were rather inadvertently introduced by the admitting physicians in all six instances—four were errors of omission and two were errors in dosing. In all six instances, the algorithm identified discrepancies at par with the clinical pharmacist. All six medication errors reached the patients involved, but none resulted in harm (National Coordinating Council for Medication Error Reporting and Prevention (NCCMERP) Index© Category C). Furthermore, a systematic review of 18 studies, and a meta-analysis of ten studies found that persistent discrepancies (discrepancies persisting after medication reconciliation) were devoid of clinical significance, with no fatal or potentially fatal outcomes (Kwan et al., 2013; Mekonnen, Abebe, Mclachlan, & Brien, 2016). The lack of clinical significance of persistent discrepancies in general, and the effectiveness of the algorithm in particular, suggests a low risk for deployment.

The efficiency and effectiveness of the algorithm in identifying discrepancies in electronic medication histories and the low-risk. involved, sets the stage for deploying an algorithmic approach to detect discrepancies and present. medication information in a standardized manner across EHR platforms and healthcare settings so as to improve medication safety and reduce medication errors. The cost savings from efficiencies that ensue in terms of time and the projected reduction in medication enors is estimated to be substantial. Furthermore, the reduced cognitive burden and time needed to detect discrepancies bears potential for engaging physicians directly and productively in the process.

### Appendix A

TABLE 1
Tabular summary of various patented and non-patented, EHR and
non-EHR approaches to reconcile medications electronically.

| | | |
|---|---|---|
| Tool | Brief description | References |

Ambulatory

| | | |
|---|---|---|
| Automated Patient | Process for patients in the waiting room of an | Lesselroth et al., 2009 |
| History Intake | ambulatory clinic to use kiosk technology to | Lesselroth et al., 2011 |
| Device (APHID) | provide their own medication histories. | |
| | US Dept. of Veterans Affairs, Portland, OR. | |

Admission and Discharge

| | | |
|---|---|---|
| Pre-Admission | Novel application designed and maintained by | Poon et al. 2006 |
| Medication List | Partners Information Systems that aggregates | Turchin et al. 2008 |
| (PAML) Builder | medication data from multiple ambulatory EHRs | Schnipper et al., 2009 |
| | (Longitudinal Medical Record and OnCall) and | |
| | custom-built inpatient computerized provider order | |
| | entry (CPOE) system | |
| | Partners HealthCare, Boston MA | |
| Web based | Web based HTML application launched from | Bails et al., 2008 |
| application | within EHR that displays a longitudinal list | |
| | including historical medications and prescriptions | |
| | generated from the EHR. | |
| | Bellevue Hospital, New York City, NY | |
| Medication | View within a commercial EHR (Eclipsys Sunrise, | Vawdrey et al., 2010 |
| reconciliation | Eclipsys Corp., Atlanta, GA) displaying two | |
| view | separate columns of inpatient and outpatient | |
| | medications. | |
| | Columbia University Medical Center, New York, | |
| | NY | |
| Electronic | Software customization in a commercial EHR | Lovins et al., 2011 |
| pathway for | (Siemens, based on workflow analysis to reconcile | |
| MedRec | medications at the transitions of care and also | |
| | generate electronic discharge instructions at a pilot | |
| | unit. | |
| | Durham Regional Hospital, Duke Univ., Durham, | |
| | NC | |
| RightRx | Computer-assisted medication reconciliation | Tamblyn et al., 2018 |
| | solution that prepopulates a community drug list | |
| | from a population-based administrative data | |
| | warehouse containing dispensed medication | |
| | records and automatically aligns it with hospital- | |
| | based medications using a combination of | |
| | clinician-focused medication sort order and | |
| | business rules. | |

Discharge

| | | |
|---|---|---|
| MOXXI | Web based prescription tool that aggregates | Tamblyn et al., 2012 |
| | medication information from the government | |
| | prescription claims system (RAMQ: Régie de | |
| | l'assurance maladie du Québec) and community | |
| | pharmacies. | |
| | McGill University Health Centre, Quebec, Canada | |
| Twinlist | Novel prototype application using JavaScript and | Markowitz et al., 2011 |
| | HTML5 using a spatial layout and multi-step | Plaisant et al., 2013 |
| | animation to visually elicit differences and | |
| | similarities between two medication lists (e.g. | |
| | intake and hospital list) and rapidly choose | |
| | medications into the reconciled list. | |
| | Univ. of Texas Health Science Center, Houston, | |
| | TX | |

Post-discharge

| | | |
|---|---|---|
| Patient Gateway | Patient portal linked to EHR allows patients to | Schnipper et al., 2008 |
| (PG) Medications | view and modify list of medications and allergies | |
| Module | from the EHR, report non-adherence, side effects | |
| | and other medication-related problems and easily | |
| | communicate this information to physicians who | |
| | can verify the information and update the EHR as | |
| | needed. | |
| | Partners HealthCare, Boston MA | |
| Partners Post | Novel application designed and maintained by | Schnipper et al., 2011 |
| Discharge | Partners Information Systems that compares the | |
| Medication | preadmission medication list to the medication list | |
| Reconciliation | generated at discharge, highlights changes and | |
| Tool | allows updates within the ambulatory EHR | |
| | (Longitudinal Medical Record). | |
| | Brigham and Women's Hospital, Boston, MA | |
| Prototype | Novel prototype application designed and | Cadwallader et al., 2013 |
| medication | maintained by Indiana University that aggregates | |
| reconciliation tool | medication information from their locally | |
| | developed EHR, pharmacies and patients in a | |
| | useful display for clinical decision making and | |
| | includes information about patient compliance. | |
| | Indiana Univ. School of Medicine, Indianapolis, | |
| | IN | |
| Secure Messaging | Enabling patients to conduct medication | Heyworth et al., 2014 |
| for Medication | reconciliation through a web portal. SMMRT used | |
| Reconciliation | to view their medications in secure email message | |
| Tool (SMMRT) | and use interactive form to verify regimens and | |
| | clarify inaccuracies. | |
| | US Dept. of Veterans Affairs, Boston, MA | |

Patented Applications

| | | |
|---|---|---|
| Cognitive | Evaluation of validity of duplicate medication | Allen, Bishop, |
| Medication | instances in aggregate patient data, and send | Chung, & Schrelber - |
| Reconciliation | notification to a computing device indicating | US 2018/0,121,606 |
| | invalidity of the duplicate instance. | (A1). |
| | International Business Machines, Armonk, NY. | |
| Medication List | Medication list is generated using scanned barcode | Schneider, Trimble, |
| Generator | information or information from a photograph | McCready & Gaul - |
| | taken with the patient's mobile device, | US 2017/0,098,060 |
| | prepopulating the list for the clinician performing | (A1). |
| | medication reconciliation. | |
| | Cerner Innovation, Inc. Kansas City, KS | |
| Medication | A mobile user device collects and transmits a | Rock, E. L. - |
| Reconciliation | medication image file to a host computer system | US 2016/0,246,928 |
| System and | that isolates individual medication elements and | (A1). |
| Method | compared against a pill image database. The | |
| | potential pill match list (and matched pairs) are | |
| | filtered, prioritized and presented for validation | |
| | producing a reconciled validated medication list. | |
| Automated Patient | Process for patients in the waiting room of an | Lesselroth, Felder, |
| History Intake | ambulatory clinic to use kiosk technology to | Adams, Cauthers, & |
| Device (APHID) | provide their own medication histories. | Wong - US |
| | US Dept. of Veterans Affairs, Portland, OR. | 2014/122129 (A1). |
| Interactive Patient | User interface technique for presenting a | Tripoli L. C. - US |
| Medication List | medication list to a patient in categories. | 2013/304500 (A1) |
| | Medimpact Healthcare Systems, Inc., San Diego | |
| | CA | |
| Maintaining | Presenting a patient with potential cost savings on | Yamaga &Tripoli - |
| Patient | a medication list reconciled from various sources | US 2011/0,184,756 |
| Medication Lists | and transmitting selected potential saving to | (A1) |
| | healthcare personnel for approval. | |
| | Medimpact Healthcare Systems, Inc., San Diego | |
| | CA | |
| Patient care | Patient care management system for monitoring | Tamblyn, Huang, |
| management | drug use by a patient, based on non-duplicate drug | Fragos, Faucher, & |
| systems and | availability data over periods of time including | Girard - US |
| methods | insufficient and over supply. The data is visually | 8,010,379 (B2) |
| | displayed in a color coded scheme on a single | |
| | screen allowing rapid assessment by a physician. | |
| | May also be adapted to assess refill compliance, | |
| | hospitalization periods, and prescription costs. | |
| | McGill University, Montreal QC | |

### Appendix B

Program Code

READ electronicMedicationHistoryResponse
// Initialize and SET all counters to zero
SET count totalMedications = 0
SET count totalDuplicateMeds = 0
SET count totalUniqueMeds = 0
SET count totalNonStdMeds = 0
// Collection of Med List, Duplicate List, Unique List, and Uncategorized List
SET <List> ( ) importedMedlist = null
SET <List> ( ) duplicateMedList = null
SET <List> ( ) uniqueMedList = null
SET <List> ( ) nonstdList =null
FOR each medication in electronicMedicationHistoryResponse

IF

(medication=“HOME_MED_COLLECTION_IMPORTED_MEDICATIONS”) THEN

add medication to importedMedlist
add related medication attributes to importedMedList
i.e CUI, Days Supply, Drug Description, NDC/RxNorm
IF (days supply is blank or days supply < 30) THEN

SET medication as acute

ELSE SET medication as chronic
END IF

sum count totalMedications + 1

END IF

END FOR
// Duplicate List
SET CUICompare1 = blank
SET CUICompare2 = blank
SET duplicatefound = false
SET count = 0
FOR each importedMed in importedMedList
CUICompare1 = importedMed CUI
count = count +1

FOR each duplicateMed in importedMedList + count

CUICompare2 = duplicateMed CUI
IF (CUICompare1=CUICompare2) THEN

add duplicateMed to duplicateMedList
sum totalDuplicateMeds +1
remove duplicateMed from importedMedList
duplicatefound=true

ELSE next duplicateMed
END IF

END FOR

IF (duplicatefound = false) THEN

add importedMed to newimportedMedList

END IF
END FOR
//Unique list and NonStandardMeds list
FOR each importedMed in newimportedMedList

IF ((importedMed drugDesc is not blank) and
(importedMed CUI is not blank)) THEN

add importedMed to uniqueMedList
sum count totaluniqueMeds+1

ELSE add imported to nonstdList

sumcount totalNonStdMeds +1

END IF

END FOR

### Appendix C

### REFERENCES

Barnsteiner, J. H. (2008). Medication Reconciliation. In R. Hughes (Author), Patient safety and quality: An evidence-based handbook for nurses. Rockville, Md.: Agency for Healthcare Research and Quality.
Cadwallader, J., Spry, K., Morea, J., Russ, A. L., Duke, J., & Weiner, M. (2013). Design of a Medication Reconciliation Application. Applied Clinical Informatics, 04(01), 110-125. doi:10.4338/aci-2012-12-ra-0057
Classen, D. C. (2003). Improving medication safety: The measurement conundrum and where to start. International Journal for Quality in Health Care, 15(90001), 41i-47. doi:10.1093/intqhc/mzg083
Cohen, M. R. (2007). Medication errors (2nd ed.). American Pharmacists Association.
Gabriel, M. H., & Swain, M. (2014, July). E-Prescribing Trends in the United States. ONC Data Brief No.18. (United States, U.S. Department of Health and Human Services, Office of the National Coordinator for Health Information Technology). Retrieved Jun. 12, 2019, from http://healthitgov.ahrqstg.org/sites/default/files/oncdatabriefe-prescribingincreases2014.pdf
Gleason, K. M., Groszek, J. M., Sullivan, C., Rooney, D., Barnard, C., & Noskin, G. A. (2004). Reconciliation of discrepancies in medication histories and admission orders of newly hospitalized patients. American Journal of Health-System Pharmacy, 61(16), 1689-1695. doi:10.1093/ajhp/61.16.1689
Gleason, K. M., Mcdaniel, M. R., Feinglass, J., Baker, D. W., Lindquist, L., Liss, D., & Noskin, G. A. (2010). Results of the Medications At Transitions and Clinical Handoffs (MATCH) Study: An Analysis of Medication Reconciliation Errors and Risk Factors at Hospital Admission. Journal of General Internal Medicine, 25(5), 441-447. doi:10.1007/s11606-010-1256-6
Greenwald, J. L., Halasyamani, L., Greene, J., Lacivita, C., Stucky, E., Benjamin, B., . . . Williams, M. V. (2010). Making inpatient medication reconciliation patient centered, clinically relevant and implementable: A consensus statement on key principles and necessary first steps. Journal of Hospital Medicine, 5(8), 477-485. doi:10.1002/jhm.849
Hasan, S., Duncan, G. T., Neill, D. B., & Padman, R. (2011). Automatic detection of omissions in medication lists. Journal of the American Medical Informatics Association, 18(4), 449-458. doi:10.1136/amiajnl-2011-000106
Heyworth, L., Paquin, A. M., Clark, J., Kamenker, V., Stewart, M., Martin, T., & Simon, S. R. (2014). Engaging patients in medication reconciliation via a patient portal following hospital discharge. Journal of the American Medical Informatics Association, 21(E1). doi:10.1136/amiajnl-2013-001995
Heyworth, L., Paquin, A. M., Clark, J., Kamenker, V., Stewart, M., Martin, T., & Simon, S. R. (2014). Engaging patients in medication reconciliation via a patient portal following hospital discharge. Journal of the American Medical Informatics Association, 21(E1). doi:10.1136/amiajnl-2013-001995
How-to Guide: Prevent Adverse Drug Events by Implementing Medication Reconciliation. Cambridge, Mass.: Institute for Healthcare Improvement; 2011.
Jagannathan, V., Mullett, C. J., Arbogast, J. G., Halbritter, K. A., Yellapragada, D., Regulapati, S., & Bandaru, P. (2009). Assessment of commercial NLP engines for medication information extraction from dictated clinical notes. International Journal of Medical Informatics, 78(4), 284-291. doi:10.1016/j.ijmedinf.2008.08.006
Kohn, L. T., Corrigan, J., & Donaldson, M. S. (2009). To err is human: Building a safer health system. National Academy Press.
Lesselroth, B., Adams, S., Felder, R., Don, D. A., Cauthers, P., Church, V., & Douglas, D. (2009). Using Consumer-Based Kiosk Technology to Improve and Standardize Medication Reconciliation in a Specialty Care Setting. The Joint Commission Journal on Quality and Patient Safety, 35(5). doi:10.1016/s1553-7250(09)35037-0
Lesselroth, B., Holahan, P., Adams, K., Sullivan, Z., Church, V., Woods, S., . . . Dorr, D. (2011). Primary care provider perceptions and use of a novel medication reconciliation technology. Journal of Innovation in Health Informatics, 19(2), 105-118. doi:10.14236/jhi.v19i2.802
Li, Z., Liu, F., Antieau, L., Cao, Y., & Yu, H. (2010). Lancet: A high precision medication event extraction system for clinical text. Journal of the American Medical Informatics Association, 17(5), 563-567. doi:10.1136/jamia.2010.004077
Lovins J, Beavers R, Lineberger R. Improving Quality, Efficiency, and Patient/provider Satisfaction with Electronic Medication Reconciliation and Discharge Instructions. Abstract published at Hospital Medicine 2011, May 10-13, Dallas, Tex. Abstract 186. Journal of Hospital Medicine. 2011; 6 (suppl 2). Accessed Apr. 16, 2019.
Manno, M. S., & Hayes, D. D. (2006). Best-Practice Interventions. Nursing, 36(3), 63-64. doi:10.1097/00152193-200603000-00045
Markowitz, E., Bernstam, E., Herskovic, J., Zhang, J., Shneiderman, B., Plaisant, C., & Johnson, T. R. (2011). Medication Reconciliation: Work Domain Ontology, Prototype Development, and a Predictive Model (pp. 878-887). AMIA Annual Symposium Proceedings. Retrieved Apr. 16, 2019.
Medication Errors and Adverse Drug Events. (2015). Retrieved from https://psnet.ahrq.gov/primers/primer/23/medication-errors
Medication Errors and Adverse Drug Events. (2015). Retrieved from https://psnet.ahrq.gov/primers/primer/23/medication-errors
National Coordinating Council for Medication Error Reporting and Prevention (NCCMERP). (2014, Nov. 20). Types of Medication Errors. Retrieved Jun. 12, 2019, from http://www.nccmerp.org/types-medication-errors
Plaisant, C., Chao, T., Wu, J., Hettinger, A., Herskovic, J. R., Johnson, T. R., Bernstam, E. V., Markowitz, E., Powsner, S., Shneiderman, B. (2013). Twinlist: Novel User Interface Designs for Medication Reconciliation (pp. 1150-1159). AMIA Annual Symposium Proceedings. Retrieved Mar. 14, 2017.
Poon, E. G., Blumenfeld, B., Hamann, C., Turchin, A., Graydon-Baker, E., Mccarthy, P. C., . . . Broverman, C. A. (2006). Design and Implementation of an Application and Associated Services to Support Interdisciplinary Medication Reconciliation Efforts at an Integrated Healthcare Delivery Network. Journal of the American Medical Informatics Association, 13(6), 581-592. doi:10.1197/jamia.m2142
Schenkel, S. (2008). The Unexpected Challenges of Accurate Medication Reconciliation. Annals of Emergency Medicine, 52(5), 493-495. doi:10.1016/j.annemergmed.2008.07.026
Schnipper, J. L., Hamann, C., Ndumele, C. D., Liang, C. L., Carty, M. G., Karson, A. S., . . . Gandhi, T. K. (2009). Effect of an Electronic Medication Reconciliation Application and Process Redesign on Potential Adverse Drug Events. Archives of Internal Medicine, 169(8), 771. doi:10.1001/archinternmed.2009.51
Schnipper, J. L., Liang, C. L., Hamann, C., Karson, A. S., Palchuk, M. B., Mccarthy, P. C., . . . Bates, D. W. (2011). Development of a tool within the electronic medical record to facilitate medication reconciliation after hospital discharge. Journal of the American Medical Informatics Association, 18(3), 309-313. doi:10.1136/amiajnl-2010-000040
Spry, K., Morea, J., Russ, A. L., Duke, J., Weiner, M., & Cadwallader, J. (2013). Design of a Medication Reconciliation Application. Applied Clinical Informatics, 04(01), 110-125. doi:10.4338/aci-2012-12-ra-0057
Surescripts. (2019). Accurate Medication History for Improved Patient Care. Retrieved Jun. 12, 2019, from http://surescripts.com/inform-care-decisions/medication-history/
Tamblyn, R., Huang, A. R., Meguerditchian, A. N., Winslade, N. E., Rochefort, C., Forster, A., . . . Reidel, K. E. (2012). Using novel Canadian resources to improve medication reconciliation at discharge: Study protocol for a randomized controlled trial. Trials, 13(1). doi:10.1186/1745-6215-13-150
Turchin, A., Hamann, C., Schnipper, J. L., Graydon-Baker, E., Millar, S. G., Mccarthy, P. C., . . . Broverman, C. A. (2008). Evaluation of an Inpatient Computerized Medication Reconciliation System. Journal of the American Medical Informatics Association, 15(4), 449-452. doi:10.1197/jamia.m2561
Using Medication Reconciliation to Prevent Errors. (2006). The Joint Commission Journal on Quality and Patient Safety, 32(4), 230-232. doi:10.1016/s1553-7250(06)32030-2
Uzuner, Ö, Solti, I., & Cadag, E. (2010). Extracting medication information from clinical text. Journal of the American Medical Informatics Association, 17(5), 514-518. doi:10.1136/jamia.2010.003947
Varkey, P., Cunningham, J., & Bisping, S. (2007). Improving Medication Reconciliation in the Outpatient Setting. The Joint Commission Journal on Quality and Patient Safety, 33(5), 286-292. doi:10.1016/s1553-7250(07)33033-x
Vawdrey, D. K., Chang, N., Compton, A., Tiase, V., & Hripcsak, G. (2010). Impact of electronic medication reconciliation at hospital admission on clinician workflow (pp. 822-826). AMIA Annual Symposium Proceedings. Retrieved Apr. 16, 2019.

### Appendix D

### Glossary

| Term | Description |
|---|---|
| Adverse events | As referenced here, limited only to adverse events that occur due to medication errors (as opposed |
| | to medical errors), and includes both potential adverse drug events (PADE) and adverse drug |
| | events (ADE) |
| Boolean (logic) | Data type named after 19^{th} century English mathematician and logician, George Boole, that has |
| | one of two possible values, usually true and false, primarily associated with conditional |
| | statements, that allow different actions by changing control flow depending on whether a |
| | programmer-specified condition is evaluated as true or false. |
| Clinical | Specialty of pharmacy that works collaboratively with practitioners and other healthcare |
| pharmacists | professionals to provide direct patient care by optimizing the use of medication, and additionally |
| | promote health, wellness, education and disease prevention. This specialty originated in hospitals |
| | and clinics, but is gradually spreading to other areas of healthcare. Most clinical pharmacists have |
| | a Doctor of Pharmacy (Pharm. D.) degree, and many have completed one or more years of post- |
| | graduate training. |
| | As referenced here, all clinical pharmacists have a Pharm. D. with post-graduate training and their |
| | role is specific to reconciling medication at admission. |
| Discrepancy | Discrepancies in medication information are unexplained, and often unintentional, differences |
| | among medications and/or their signature information, documented across various healthcare |
| | settings, and what a patient may actually be taking. Specifically, these discrepancies from |
| | incomplete or inaccurate medication information lead to medication errors that are preventable. |
| Electronic | Often used interchangeably with Electronic Medical Record (EMR), due to the prevalent |
| Health Record | ambiguity. |
| (EHR) | An EMR is an application used by healthcare organizations to document, monitor, and manage |
| | healthcare delivery. It includes technological tools (e.g. Clinical Decision Support Systems), and |
| | is the legal record of what happened to the patient during their encounter. An EMR is owned by |
| | the healthcare organization (despite ownership, patients have rights to access their information |
| | within an EMR). |
| | An EHR is a subset of an EMR that provides clinical information regarding a patient and |
| | processes real-time transactions (e.g. producing clinical summaries). It is owned by the patient |
| | and can incorporate patient input (through portals). Provides access to other episodes of care at |
| | other healthcare organizations through Health Information Exchanges and can similarly, transmit |
| | information. |
| Healthcare | Acute: hospital (in-patient) |
| settings | Ambulatory: clinic, office (out-patient) |
| | Long-term care: assisted living, nursing home etc. |
| Medication | Name (chemical, brand, and generic), and signature information (dosage, frequency and route of |
| information | administration) of a drug. |
| Medication | As reference here, limited to ‘preventable’ medication errors that occur due to discrepancies from |
| error | incomplete and inaccurate medication information. Typically such errors lead to errors of |
| | omission and commission that result in duplication, sub-optimal dosing, and drug interactions. |
| | In the broader context, medication errors are technically defined as “any preventable event that |
| | may cause or lead to inappropriate medication use or patient harm while the medication is in the |
| | control of a healthcare professional, patient, or consumer. Such events may be related to |
| | professional practice, healthcare products, procedures, and systems, including prescribing, order |
| | communication, product labeling, packaging, nomenclature, compounding, dispensing, |
| | distribution, administration, education, monitoring, and use.” |
| Medication | Process of compiling the most current, accurate and complete list of all prescription and over-the |
| reconciliation | counter medications that a patient may be taking comprising of the following essential steps: |
| | verification of name (chemical, brand, and generic) and signature information (dosage, |
| | frequency and route of administration) of a drug(s). |
| | clarification of compliance, appropriateness of the indication and dosage for the stated |
| | diagnoses. |
| | reconciliation of any discrepancies identified during this process. |
| | A sub-process that is often included as a step, is onward and effective communication of this |
| | reconciled list with the patient, other caregivers, health information exchanges etc. |
| National Drug | he most prevalent numeric drug identifier terminology scheme used in electronic prescriptions |
| Code (NDC) | from the Food and Drug Administration, originally issued for tracking purposes in 1972. Hence a |
| | single clinical drug concept (drug name, strength, and dosage form) may have multiple NDC |
| | identifiers to account for various manufacturers, packaging sizes and product forms (liquid, solid |
| | etc.). Multiple identifiers for a drug form (For e.g. Amoxicillin 500 mg oral capsule has at least |
| | 227 distinct NDC codes without any intrinsic characteristics linking them), regional availability, |
| | and lack of an updated, authoritative database for matching and cross-referencing, all make the |
| | use of NDC identifiers cumbersome, restrictive and confusing, thereby limiting its potential to be |
| | used as a preferred terminology system in electronic prescriptions (Nelson, Zeng, Kilbourne, |
| | Powell, & Moore, 2011). |
| Pharmacy | Third-party administrator of prescription drug programs for commercial health plans, employer |
| Benefit | plans, federal and state plans, and health benefit programs. PBMs are primarily responsible for |
| Managers | developing and maintaining formularies, contracting with pharmacies, negotiating discounts and |
| (PBM) | rebates with manufacturers, and processing and paying prescription drug claims; with the goal of |
| | maintaining or reducing pharmacy expenditures for participating plans and programs, while trying |
| | to improve outcomes. |
| Physician | Technically, the term (healthcare) ‘practitioner’ includes physicians (doctor of medicine (MD) or |
| | osteopathy (DO)), Nurse Practitioners (NP), Advanced practice nurses (APNs), physician |
| | assistants (PA), nurse-midwives, podiatrist, dentists, chiropractors, clinical psychologists, |
| | optometrists etc. As referenced here, the term physician intends to describe the prescribing role |
| | and avoid overlapping connotations with the terms practitioner and providers. |
| RxNorm | RxNorm is a freely available, non-proprietary, standardized numeric drug identifier terminology |
| | developed by the United States National Library of Medicine (NLM) in 2002 within the larger |
| | Unified Medical Language Systems (UMLS) project that is updated on a monthly basis. RxNorm |
| | normalizes names for generic and branded drugs and supports semantic interoperability between |
| | drug terminologies and pharmacy knowledgebase systems by grouping similar drugs into |
| | concepts that are assigned a normalized name consisting of the ingredient, strength, and dose form |
| | (in that order) and an RxNorm concept unique identifier that shares the same meaning at a certain |
| | level of abstraction. Each concept is (RxCUI) is machine readable, is never deleted or reused, and |
| | the meaning persists across releases. Concepts can also include relationships to other attributes |
| | such as NDCs, marketing categories, and pill imprint information. RxNorm is being increasingly |
| | used for electronic prescriptions, and overtime, may supersede NDC in nomenclature and |
| | terminology based utilities. |
| Signature | Standard part of a prescription that specify directions for use (dosage, frequency and route of |
| information | administration). Often abbreviated as sig (from Latin Signa - label), not to be confused with sig |
| | codes (e.g. B.I.D. - take twice daily). |
| Non-standard | Refers to the sorting of medications by the algorithm and clinical pharmacist. From the electronic |
| discrepancies | medication history response, medications that do not bear standard numeric drug identifiers such |
| | as over-the-counter food supplements (e.g. protein shakes), supplies (e.g. glucose monitor), |
| | immunizations etc. and may not be technically defined as drugs by the FDA. |
| Unique | Refers to the sorting of medications by the algorithm and clinical pharmacist. From the electronic |
| medications | medication history response, medications that are not duplicates, and bear standard numeric drug |
| | identifiers that distinguishes them from supplies, immunizations etc. |

The phrases and terminology used to describe the invention and embodiment are primarily intended to convey the principle(s), practical application(s) and technical improvement(s) over current methods and processes of the invention to those of ordinary skill. This description is neither meant to be exhaustive or comprehensive, nor limited to the execution of a particular, or plurality of feature(s) or element(s) of the embodiment. Additional applications, technical improvements, and forms may be apparent to those of ordinary skill in this or other arts, without significant departure from the principle and spirit of the invention and embodiment, with or without specific modifications for the utility contemplated.

What is claimed is:

1. A method, when used in a data processing system comprising of at least one processor and at least one memory, bearing instructions to execute operations comprising:
a. Reading of aggregate medication data, by the data processing system, obtained from computing devices associated with a plurality of different sources and data formats of electronic medication histories for a patient;
b. Analysis of aggregate medication data, by the data processing system, to identify discrepancies in medications by analyzing the content of every instance of the aggregate medication data, further comprising of—
Determination that every instance of the aggregate medication data, by the data processing system, is indeed a medication as defined by the Food and Drug Administration (FDA) by its standard numeric drug identifiers (NDC/RxNorm—also please see glossary), whereas instances lacking standard numeric drug identifiers are categorized as non-standard discrepancies;
Determination that every instance of the aggregate medication data, by the data processing system, is indeed a medication as defined by the Food and Drug Administration (FDA) by its standard numeric drug identifiers (NDC/RxNorm—please also see glossary), whereas instances known to be either an immunization or supply (e.g. glucometer) are identified and categorized as immunization and supplies;
Determination that every instance of the aggregate medication data, by the data processing system, is non-duplicative, whereas duplicate instances including partial string matches or evolving signature information (please also see [0003], [0013], and glossary) are identified and categorized as duplicate discrepancies;
Determination that every instance of the aggregate medication data, by the data processing system, is non-duplicative, whereas unique (non-duplicate) instances are identified and further categorized as either acute or chronic depending on whether the prescription was issued for greater than 30 days and/or refills authorized;
c. Generation of sorted and categorized medication list with discrepancies identified, by the data processing system, obtained from reading and analysis of aggregate medication data from computing devices associated with a plurality of different sources and data formats of electronic medication histories for a patient as outlined above, such that it can be presented within the electronic medication history response in a format that improves medication safety and reduction of medication errors;

2. A process to improve medication safety and medication management, based on the method of claim 1, wherein the operation executed, by the data processing system, analyzes the content of every instance of the aggregate medication data in electronic medication histories, further comprising of—
a. Generation of sorted electronic medication history response with discrepancies identified in standardized categories of discrepant and non-discrepant medications consistently across EHR platforms so as to promote medication reconciliation and reduce medication errors across healthcare settings.
b. Reduce the cognitive burden and time needed to detect and reconcile discrepant medications so as to engage clinicians, and derive efficiencies and cost savings.
Furthermore, insights from the method of claim 1 and the process of claim 2 may facilitate homogenous exchange of medication information across EHR platforms and healthcare settings through three key domains of healthcare policy—(a) mandates or incentives for importing an electronic medication history prior to medication reconciliation, prescription of new medications, and renewal of chronic prescriptions, (b) mandates or incentives for information exchange across all electronic prescription networks including ‘self-contained (closed)’ networks such as Kaiser Permanente (Gabriel and Swain, 2014), and (c) mandates or incentives for the universal adoption of standard contemporary numeric drug identifiers, namely RxNorm (see glossary) that is continuously updated and maintained.
